(19) United States
(12) Patent Application Publication
(10) Pub. No.: US 2025/0166091 A1
(43) Pub. Date: May 22, 2025

(54) SYSTEMS, DEVICES AND METHODS FOR MANAGING A HERD OF LIVESTOCK

(71) Applicant: Cattleytics Inc., Hamilton (CA)

(72) Inventors: Sharon Louise van de Pol, Hamilton (CA); John Thomas Clark, Hamilton (CA)

(21) Appl. No.: 18/954,417

(22) Filed: Nov. 20, 2024

Related U.S. Application Data
(60) Provisional application No. 63/601,007, filed on Nov. 20, 2023.

(51) Int. Cl. G06Q 50/02; G06F 30/13; G06Q 10/0631; G16H 20/00; G16H 50/70

(52) U.S. Cl. CPC G06Q 50/02; G06F 30/13; G16H 50/70; G06Q 10/06316; G16H 20/00

(57) ABSTRACT

Systems, devices and methods for managing a herd are described herein. The systems include a processor configured to receive livestock data from a herd management software system. The herd management system is configured to receive source data from each data source of a plurality of data sources. The source data includes health data for the herd of livestock. The herd management system is also configured to normalize the source data received from each data source of the plurality of data sources to link the source data from each of the data sources for a specified livestock of the herd and generate the livestock data. The processor is also configured to create a data model based on the livestock data. The data model identifies a trend in the health data of the herd of livestock. The processor is also configured to, based on the data model and the identified trend, generate a task for managing the herd of livestock.

### RELATED APPLICATIONS

The present application claims priority to U.S. Provisional Patent Application No. 63/601,007 entitled “SYSTEMS, DEVICES AND METHODS FOR MANAGING A HERD OF LIVESTOCK” filed on Nov. 20, 2023, the entire contents of which are hereby incorporated by reference herein.

### TECHNICAL FIELD

This disclosure relates generally to systems, devices and methods for managing a herd, and more specifically to systems, devices and methods for managing a herd of livestock.

### BACKGROUND

Dairy farmers face massive barriers to carrying out business in ways that most industries take for granted. Even a simple, automated income over cost calculation is not easily workable in the industry. Farmers who would like to change the trajectory of their production—whether it is due to finances, methane capture or optimizing some other value—typically rely on complicated manual calculations, or else cannot rely on numerical data at all to support farm planning. Dairy farming also requires expertise in a wide range of domains, including management, reproduction, economics, nutrition, animal health, pharmaceuticals, and milk quality. Moreover, they must effectively convey their decision-making processes, knowledge, and skills to a dwindling dairy workforce.

Creating a clear business and implementation plan for improvement or modernization can seem insurmountable when so many variables affect a farmer's success. Currently, nearly half of all dairy farms use no management software, losing out on potential efficiency-based revenues that other industries take for granted. As much as historical dairy farming is romanticized, modern consumers and regulators are also demanding efficiencies tied to a farm's carbon footprint that cannot be managed reasonably without some form of digitization.

Dairy farmers who opt for digitization often discover that it adds to their workload rather than easing it. Instead of making decisions easier and allowing for individualized care for each animal, information to help with larger economic decisions cannot be extracted from the tools currently available on the market. Connecting or trending data can be difficult at best, and impossible at worst.

The aforementioned struggles are not limited to dairy farmers either. Farmers managing herds of other livestock face similar issues.

Accordingly, there is a need for improved systems, devices and methods for managing a herd of livestock.

### SUMMARY

In accordance with a broad aspect, a system for managing a herd of livestock is described herein. The system includes a memory and a processor in communication with the memory. The processor is configured to receive livestock data from a herd management software system. The herd management system is configured to receive source data from each data source of a plurality of data sources. The source data includes at least one of health data, production data, growth data and genomic data for the herd of livestock. The herd management system is also configured to normalize the source data received from each data source of the plurality of data sources to link the source data from each of the data sources for a specified livestock of the herd and generate the livestock data. The processor is also configured to create a data model based on the livestock data, the data model identifying a trend in the health data of the herd of livestock. The processor is also configured to generate, based on the data model and the identified trend, a task for managing the herd of livestock and output the task.

In at least one embodiment, the health data includes cow calendar data for each livestock of the herd.

In at least one embodiment, the generated task is a treatment plan for one or more specific livestock of the herd or a vaccine administration timeline.

In at least one embodiment, the data model includes a personalized timeline for each livestock in the herd, the personalized timeline including past events during the life of the livestock including at least one of: birth date, date bred, date of lameness, date of foot trim, date of pregnancy, and date of vaccination.

In at least one embodiment, the personalized timeline includes one or more dates of predicted events, the predicted events including one or more of a date of giving birth and/or a lactation period start date and/or a lactation period end date, the predicted events being determined based on the health data.

In at least one embodiment, the livestock data includes production data and the data model includes a model of milk production on the farm.

In at least one embodiment, the processor is further configured to, after generating the task for managing the herd of livestock, generating a protocol for completing the task.

In at least one embodiment, the protocol includes video instructions, audio instructions and/or written instructions for completing the task.

In at least one embodiment, the protocol is pre-populated by an operator of the processor.

In at least one embodiment, the protocol is at least partially provided by the user by uploading at least one of the video instructions, the audio instructions and/or the written instructions to the processor before the processor generates the task.

In at least one embodiment, the processor is further configured to: receive image data depicting at least a portion of a farm; receive a user input indicating a boundary of one or more selected geographical areas of the farm; receive a selection of one or more livestock of the herd, the one or more livestock having specified livestock data associated therewith stored on the memory; and assign the one or more livestock to the selected geographical area of the farm.

In at least one embodiment, the specified livestock data includes location data providing a geographical location of the livestock.

In at least one embodiment, the image data is one of an aerial image, an AutoCAD drawing and mapping data received from a third party data source.

In at least one embodiment, the processor is further configured to display the specified livestock data on a user interface depicting the boundary on the image data, the specified livestock data overlaying the image data within the boundary.

In at least one embodiment, the task includes a treatment regime for an illness of the herd, the illness being identified based on the identified trend.

In at least one embodiment, the treatment regime includes a suggestion of a drug to administer to the livestock to treat the illness, the suggestion based on the health data, the health data including at least one of: a temperature of the livestock, a weight of the livestock, a gender of the livestock and an age of the livestock.

In at least one embodiment, the treatment regime includes a dosage route of the drug and/or a physical location of the drug on the farm.

In at least one embodiment, the treatment regime includes an inventory of the drug on the farm.

In at least one embodiment, the system also includes a display device centrally located on the farm, the display device presenting the task to be performed to manage the herd of livestock.

A computer implemented method of managing a herd of livestock is also described herein. The computer implemented methods includes: receiving, at a processor, livestock data from a herd management software system, the herd management system configured to: receive source data from each data source of a plurality of data sources, the source data including health data for the herd of livestock; and normalize the source data received from each data source of the plurality of data sources to link the source data from each of the data sources for a specified livestock of the herd and generate the livestock data. The method also includes creating, by the processor, a data model based on the livestock data, the data model identifying a trend in the health data of the herd of livestock; generating, by the processor, based on the data model and the identified trend, a task for managing the herd of livestock; and outputting the task.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows a system diagram in accordance with one or more embodiments described herein.

FIG. 2 shows a device diagram in accordance with one or more embodiments described herein.

FIG. 3A shows a server diagram in accordance with one or more embodiments described herein.

FIG. 3B shows a diagram of integration of third party data into the systems and devices interface in accordance with one or more embodiments described herein.

FIG. 4A shows an exemplary user interface in accordance with one or more embodiments described herein.

FIGS. 4B and 4C show example images that user can use to associate data with specific areas of the images.

FIGS. 5A, 5B, 5C, 5D, 5E and 5F show exemplary user interfaces in accordance with one or more embodiments described herein.

FIGS. 6A, 6B and 6C show exemplary user interfaces in accordance with one or more embodiments described herein.

FIG. 6D shows exemplary milk quantity data by days-in-milk (DIM) determined using the polynomial equation provided therein.

FIG. 7A shows an exemplary user interface in accordance with one or more embodiments described herein.

FIG. 7B shows a display showing an exemplary user interface in accordance with one or more embodiments described herein.

Further aspects and features of the example embodiments described herein will appear from the following description taken together with the accompanying drawings.

### DESCRIPTION OF VARIOUS EMBODIMENTS

Various systems, devices and methods are described below to provide an example of at least one embodiment of the claimed subject matter. No embodiment described below limits any claimed subject matter and any claimed subject matter may cover systems, devices and methods that differ from those described below. The claimed subject matter are not limited to systems, devices and methods having all of the features of any one systems, device or method described below or to features common to multiple or all of the systems, devices and methods described below. It is possible that a system, device or method described below is not an embodiment of any claimed subject matter. Any subject matter that is disclosed in a system, device or method described herein that is not claimed in this document may be the subject matter of another protective instrument, for example, a continuing patent application, and the applicant(s), inventor(s) and/or owner(s) do not intend to abandon, disclaim, or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

It should be noted that terms of degree such as “substantially”, “about” and “approximately” as used herein mean a reasonable amount of deviation of the modified term such that the end result is not significantly changed. These terms of degree should be construed as including a deviation of the modified term, such as 1%, 2%, 5%, or 10%, for example, if this deviation does not negate the meaning of the term it modifies.

As used herein and in the claims, two or more parts are said to be “coupled”, “connected”, “attached”, “joined”, “affixed”, or “fastened” where the parts are joined or operate together either directly or indirectly (i.e., through one or more intermediate parts), so long as a link occurs. As used herein and in the claims, two or more parts are said to be “directly coupled”, “directly connected”, “directly attached”, “directly joined”, “directly affixed”, or “directly fastened” where the parts are connected in physical contact with each other. As used herein, two or more parts are said to be “rigidly coupled”, “rigidly connected”, “rigidly attached”, “rigidly joined”, “rigidly affixed”, or “rigidly fastened” where the parts are coupled so as to move as one while maintaining a constant orientation relative to each other. None of the terms “coupled”, “connected”, “attached”, “joined”, “affixed”, and “fastened” distinguish the manner in which two or more parts are joined together.

Furthermore, the recitation of any numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation up to a certain amount of the number to which reference is being made, such as 1%, 2%, 5%, or 10%, for example, if the end result is not significantly changed.

The following description is not intended to limit or define any claimed or as yet unclaimed subject matter. Subject matter that may be claimed may reside in any combination or sub-combination of the elements or process steps disclosed in any part of this document including its claims and figures. Accordingly, it will be appreciated by a person skilled in the art that an apparatus, system or method disclosed in accordance with the teachings herein may embody any one or more of the features contained herein and that the features may be used in any particular combination or sub-combination that is physically feasible and realizable for its intended purpose.

The example systems and methods described herein may be implemented in hardware or software, or a combination of both. In some cases, the examples described herein may be implemented, at least in part, by using one or more computer programs, executing on one or more programmable devices comprising at least one processing element, a data storage element (including volatile and non-volatile memory and/or storage elements), and at least one communication interface. These devices may also have at least one input device (e.g. a keyboard, a mouse, a touchscreen, and the like), and at least one output device (e.g. a display screen, a printer, a wireless radio, and the like) depending on the nature of the device. For example, and without limitation, the programmable devices (referred to below as computing devices) may be a server, network appliance, embedded device, computer expansion module, a personal computer, laptop, personal data assistant, cellular telephone, smart-phone device, tablet computer, a wireless device or any other computing device capable of being configured to carry out the methods described herein.

In some examples, the communication interface may be a network communication interface. In examples in which elements are combined, the communication interface may be a software communication interface, such as those for inter-process communication (IPC). In still other examples, there may be a combination of communication interfaces implemented as hardware, software, and a combination thereof.

Program code may be applied to input data to perform the functions described herein and to generate output information. The output information is applied to one or more output devices, in known fashion.

Each program may be implemented in a high-level procedural, declarative, functional or object-oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Examples of the system may also be considered to be implemented as a non-transitory computer-readable storage medium, configured with a computer program, where the storage medium so configured causes a computer to operate in a specific and predefined manner to perform the functions described herein.

Furthermore, the example system, processes and methods are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloads, magnetic and electronic storage media, digital and analog signals, and the like. The computer useable instructions may also be in various forms, including compiled and non-compiled code.

Various examples of systems, methods and computer programs products are described herein. Modifications and variations may be made to these examples without departing from the scope of the invention, which is limited only by the appended claims. Also, in the various user interfaces illustrated in the figures, it will be understood that the illustrated user interface text and controls are provided as examples only and are not meant to be limiting. Other suitable user interface elements may be used with alternative implementations of the systems and methods described herein.

As used herein, the term “user” refers to a user of a user device, and the term “subject” refers to a subject whose measurements are being collected. The user and the subject may be the same person, or they may be different persons in the case where one individual operates the user device and another individual is the subject. For example, in one embodiment the user may be a health care professional such as a nurse, doctor or dietitian and the subject is a human patient.

Recently, there has been a growing interest in developing new systems, devices and methods for managing a herd of livestock.

The various embodiments described herein generally relate to systems, devices and methods for managing a herd of livestock.

More specifically, the systems, devices and methods for managing a herd of livestock described herein are designed for integrating with and/or replacing existing herd management software.

More specifically, on a typical farm, such as but not limited to a dairy farm, there are a many data sources that operate in a disconnected fashion making it almost impossible to model the most basic economic data. The systems, devices and methods described herein provide a modelling framework for predictive management of farms.

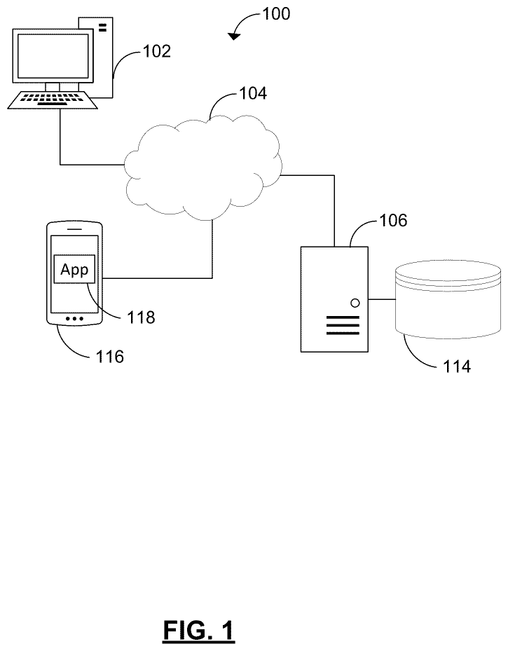

Reference is first made to FIG. 1, which shows a system diagram 100 of a management system for a herd of livestock. The system 100 includes one or more computer devices 102, a network 104, one or more servers 106, one or more data stores 114, and one or more user devices 116.

The one or more computer devices 102 may be used by a user such as a government official, an industry partner, a veterinarian, a farmer, manager, administrator or other farming professional to access a software application (not shown) running on server 106 over network 104. In one embodiment, the one or more computer devices 102 may access a web application hosted at server 106 using a browser for reviewing farm data, for example livestock data entered by users using user devices 116 and/or livestock data uploaded from a third party data storage. Farm data may also include location data, etc. The one or more user devices 116 may download an application 118 (including downloading from an App Store such as the Apple® App Store or the Google® Play Store) for entering and/or viewing farm data through server 106.

The one or more user devices 116 may be any two-way communication device with capabilities to communicate with other devices. A user device 116 may be a mobile device such as mobile devices running the Google® Android® operating system or Apple® iOS® operating system. A user device 116 may be a smart speaker, such as an Amazon® Alexa® device, or a Google® Home® device. A user device 116 may be a smart watch such as the Apple® Watch, Samsung® Galaxy® watch, a Fitbit® device, or others as known.

A user device 116 may be the personal device of a user, or may be a device provided by an employer. The one or more user devices 116 may be used by an end user to access the software application (not shown) running on server 106 over network 104. In one embodiment, the one or more user devices 116 may access a web application hosted at server 106 using a browser for managing a herd of livestock. In an alternate embodiment, the one or more user devices 116 may download an application 118 (including downloading from an App Store such as the Apple® App Store or the Google® Play Store) for managing a herd of livestock. The user device 116 may be a desktop computer, mobile device, or laptop computer. The user device 116 may be in communication with server 106, and may allow a user to review a user profile stored in a database at data store 114 farming data. The users using user devices 116 may provide farming data using a software application. The application 118 may be an app for managing a herd of livestock.

The software application running on the one or more user devices 116 may display one or more user interfaces on a display device of the user device 116.

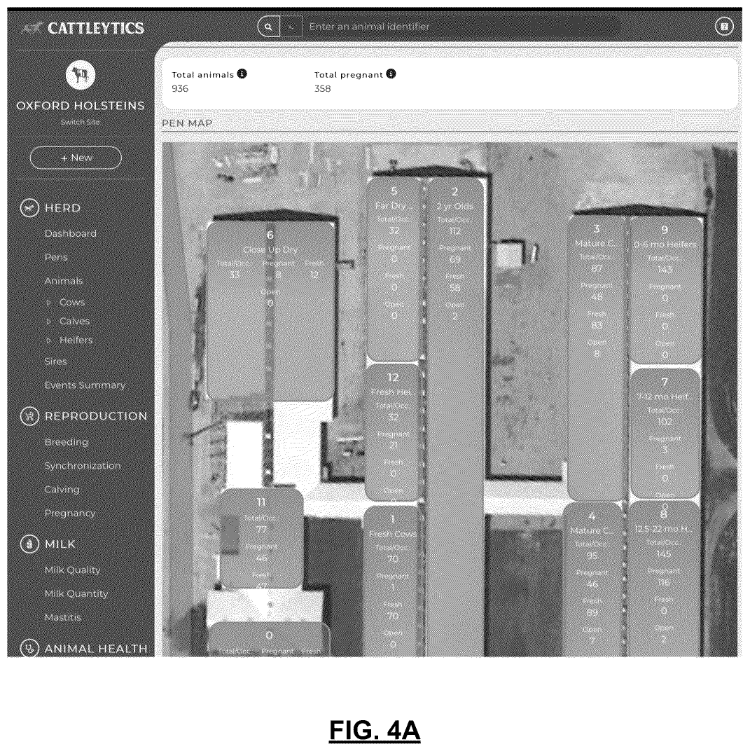

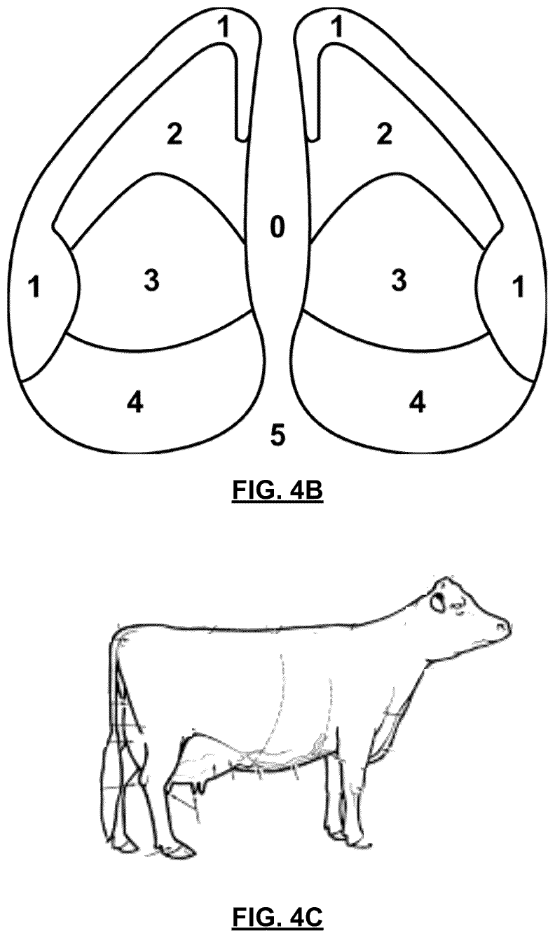

The software application running on the one or more computer devices 102 may display one or more user interfaces on a display device of the computer device 102, including, but not limited to, the various user interfaces shown in FIGS. 4A-7C.

Network 104 may be any network or network components capable of carrying data including the Internet, Ethernet, fiber optics, satellite, mobile, wireless (e.g. Wi-Fi, WiMAX), SS7 signaling network, fixed line, local area network (LAN), wide area network (WAN), a direct point-to-point connection, mobile data networks (e.g., Universal Mobile Telecommunications System (UMTS), 3GPP Long-Term Evolution Advanced (LTE Advanced), Worldwide Interoperability for Microwave Access (WiMAX), etc.) and others, including any combination of these.

The server 106 is in network communication with the one or more user devices 116 and the one or more computer devices 102. The server 106 may further be in communication with a database at data store 114. The database at data store 114 and the server 106 may be provided on the same server device, may be configured as virtual machines, or may be configured as containers. The server 106 and a database at data store 114 may run on a cloud provider such as Amazon® Web Services (AWS®).

The server 106 may host a web application or an Application Programming Interface (API) endpoint that the one or more user devices 116 may interact with via network 104. The server 106 may make calls to the mobile device 116 to poll for farming data. Further, the server 106 may make calls to the database at data store 114 to query subject data, subject model data, or other data received from the users of the one or more user devices 116. The requests made to the API endpoint of server 106 may be made in a variety of different formats, such as JavaScript Object Notation (JSON) or extensible Markup Language (XML). The farming data may be transmitted between the server 106 and the user device 116 and/or the computer device 102 in a variety of different formats. The farming data received by the data store 114 from the one or more user devices 116 and/or the computer device 102 may be stored in the database at data store 114, or may be stored in a file system at data store 114. The file system may be a redundant storage device at the data store 114, or may be another service such as Amazon® S3, or Dropbox.

The database of data store 114 may store subject information including name, birth date, age, gender, etc. of the subjects (i.e. livestock). The database of data store 114 may be a Structured Query Language (SQL) such as PostgreSQL or MySQL or a not only SQL (NoSQL) database such as MongoDB.

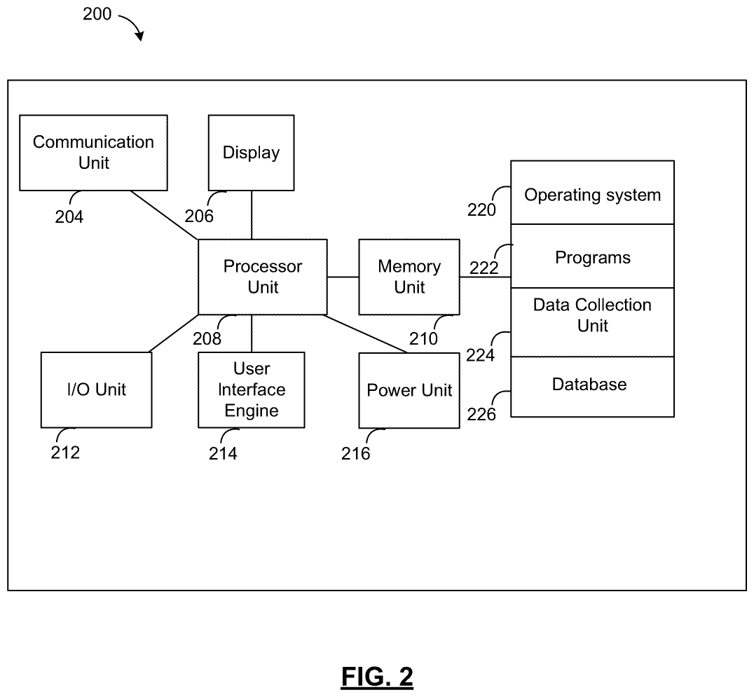

Referring next to FIG. 2 there is shown another system diagram 200 of an alternate embodiment of the T2DM prediction system for model generation. The one or more computer devices 202, the network 204, the one or more user devices 216, the server 206, and the data store 214 generally correspond to the one or more computer devices 102, the network 104, the one or more user devices 116, the server 106, and the data store 114 respectively of FIG. 1.

FIG. 2 shows a user device diagram 200 showing detail of the one or more user devices 116 in FIG. 1. It should be understood that the components described below are common to the user devices 116 described above as well as the computer device 102, each of which are configured to run an application for managing a herd of livestock including sending and receiving farm data to server 106.

The user device 116 includes one or more of a communication unit 204, a display 206, a processor unit 208, a memory unit 210, I/O unit 212, a user interface engine 214, a power unit 216. The user device 116 may be a laptop, gaming system, smart speaker device, mobile phone device, smart watch or others as are known. The user device 116 may be a passive sensor system proximate to the user, for example, a device worn on user, or on the clothing of the user.

The communication unit 204 can include wired or wireless connection capabilities. The communication unit 204 can include a radio that communicates utilizing CDMA, GSM, GPRS or Bluetooth protocol according to standards such as IEEE 802.11a, 802.11b, 802.11g, or 802.11n. The communication unit 204 can be used by the mobile device 116 to communicate with other devices or computers.

Communication unit 204 may communicate with the wireless transceiver 318 to transmit and receive information via local wireless network. The communication unit 304 may provide communications over the local wireless network using a protocol such as Bluetooth (BT) or Bluetooth Low Energy (BLE).

The display 206 may be an LED or LCD based display, and may be a touch sensitive user input device that supports gestures.

The processor unit 208 controls the operation of the mobile device 116. The processor unit 208 can be any suitable processor, controller or digital signal processor that can provide sufficient processing power depending on the configuration, purposes and requirements of the user device 116 as is known by those skilled in the art. For example, the processor unit 208 may be a high performance general processor. In alternative embodiments, the processor unit 208 can include more than one processor with each processor being configured to perform different dedicated tasks. In alternative embodiments, it may be possible to use specialized hardware to provide some of the functions provided by the processor unit 208. For example, the processor unit 208 may include a standard processor, such as an Intel® processor, an ARM® processor or a microcontroller.

The processor unit 208 can also execute a user interface (UI) engine 214 that is used to generate various UIs, some examples of which are shown and described herein.

The present systems, devices and methods may provide an improvement in the operation of the processor unit 208 by ensuring the analysis of livestock data is performed using accurate information relating to individual subjects (i.e. livestock member) and/or individual subjects of defined geographic locations. The reduced processing required for the individual subjects in the analysis (as compared with processing the superset of all subjects) reduces the processing burden required to make predictions based on livestock data.

The memory unit 210 comprises software code for implementing an operating system 220 and programs 222. The memory unit 210 can include RAM, ROM, one or more hard drives, one or more flash drives or some other suitable data storage elements such as disk drives, etc. The memory unit 210 is used to store an operating system 220 and programs 222 as is commonly known by those skilled in the art.

The I/O unit 212 can include at least one of a mouse, a keyboard, a touch screen, a thumbwheel, a track-pad, a track-ball, a card-reader, an audio source, a microphone, voice recognition software and the like again depending on the particular implementation of the user device 116. In some cases, some of these components can be integrated with one another.

The user interface engine 214 is configured to generate interfaces for users to view farm data, view analyses of the farm data, view model predictions, etc. The various interfaces generated by the user interface engine 214 are displayed to the user on display 206.

The power unit 216 can be any suitable power source that provides power to the user device 116 such as a power adaptor or a rechargeable battery pack depending on the implementation of the user device 116 as is known by those skilled in the art.

The operating system 220 may provide various basic operational processes for the user device 116. For example, the operating system 220 may be a mobile operating system such as Google® Android® operating system, or Apple® iOS® operating system, or another operating system.

The programs 222 include various user programs so that a user can interact with the user device 116 to perform various functions. The programs 222 may include a farm management application. The farm management application may be configured for many farm related activities, such as, but not limited to, viewing farm data, such as but not limited to livestock data, receiving and sending farm data, receiving any other data related to farm management, as well as receiving messages, notifications and alarms as the case may be. The programs 222 may include a telephone calling application, a voice conferencing application, social media applications, and other applications as known.

The data collection unit 224 receives farm data, either manually input by a user of device 116 or automatically collected using one or more programs 222.

In one or more embodiments, the data collection unit 224 receives medical data, or other clinical data and may store it in database 226. The data collection unit 224 may receive the clinical data and may transmit it to a server. The data collection unit 224 may supplement the clinical data that is received from the user of the device 116 with mobile device data and mobile device metadata.

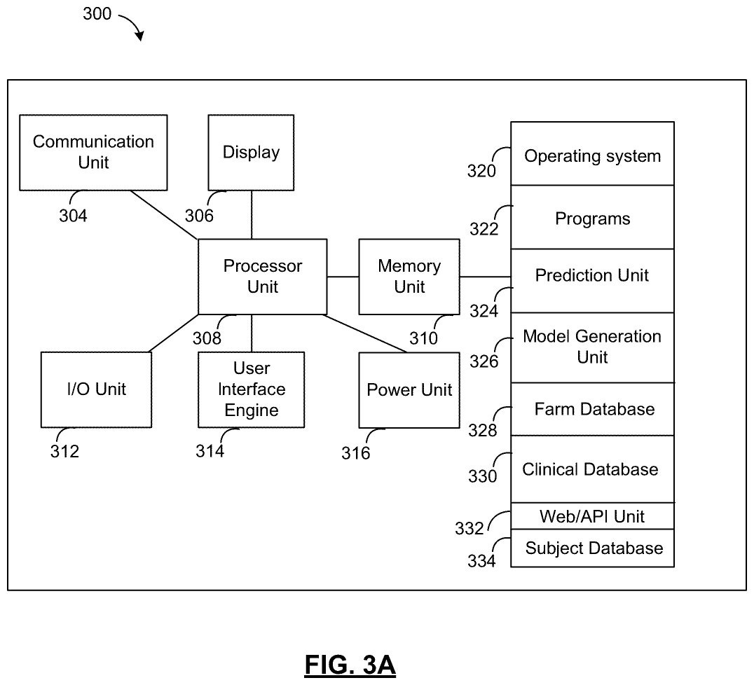

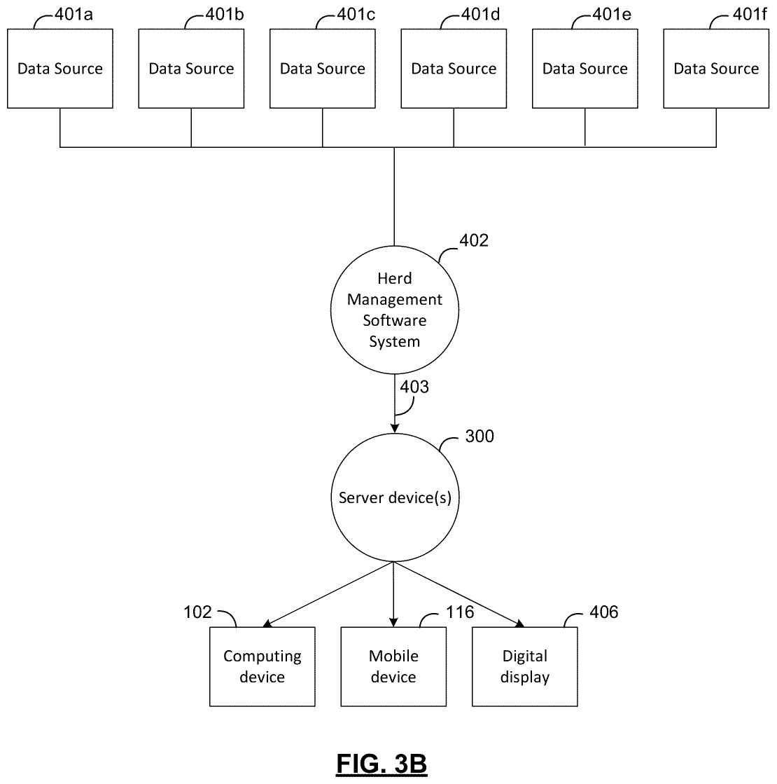

FIG. 3A shows a server diagram showing detail of the server 106 in FIG. 1. The server 300 includes one or more of a communication unit 304, a display 306, a processor unit 308, a memory unit 310, I/O unit 312, a user interface engine 314, and a power unit 316.

The communication unit 304 can include wired or wireless connection capabilities. The communication unit 304 can include a radio that communicates using standards such as IEEE 802.11a, 802.11b, 802.11g, or 802.11n. The communication unit 304 can be used by the server 300 to communicate with other devices or computers. Communication unit 304 may communicate with a network, such as network 104 (see FIG. 1).

The display 306 may be an LED or LCD based display, and may be a touch sensitive user input device that supports gestures. Display 306 may also be any other display known to those skilled in the art.

The processor unit 308 controls the operation of the server 300. The processor unit 308 can be any suitable processor, controller or digital signal processor that can provide sufficient processing power depending on the configuration, purposes and requirements of the server 300 as is known by those skilled in the art. For example, the processor unit 308 may be a high performance general processor. In alternative embodiments, the processor unit 308 can include more than one processor with each processor being configured to perform different dedicated tasks. The processor unit 408 may include a standard processor, such as an Intel® processor or an AMD® processor.

The processor unit 308 can also execute a user interface (UI) engine 314 that is used to generate various UIs for delivery via a web application provided by the Web/API Unit 330, some examples of which are shown and described herein, such as interfaces shown in FIGS. 4A-7C.

The memory unit 310 comprises software code for implementing an operating system 320, programs 322, prediction unit 324, model generation unit 326, farm database 328, clinical database 330, Web/API Unit 332, and subject database 334.

The memory unit 310 can include RAM, ROM, one or more hard drives, one or more flash drives or some other suitable data storage elements such as disk drives, etc. The memory unit 310 is used to store an operating system 320 and programs 322 as is commonly known by those skilled in the art.

The I/O unit 312 can include at least one of a mouse, a keyboard, a touch screen, a thumbwheel, a track-pad, a track-ball, a card-reader, an audio source, a microphone, voice recognition software and the like again depending on the particular implementation of the server 300. In some cases, some of these components can be integrated with one another.

The user interface engine 314 is configured to generate interfaces for users to configure voice measurement, record training voice data, view T2DM prediction data, view voice sample data, view T2DM prediction, etc. The various interfaces generated by the user interface engine 314 may be transmitted to a user device by virtue of the Web/API Unit 332 and the communication unit 304.

The power unit 316 can be any suitable power source that provides power to the server 300 such as a power adaptor or a rechargeable battery pack depending on the implementation of the server 300 as is known by those skilled in the art.

The operating system 320 may provide various basic operational processes for the server 300. For example, the operating system 320 may be a server operating system such as Ubuntu® Linux, Microsoft® Windows Server® operating system, or another operating system.

The programs 322 include various user programs. They may include several hosted applications delivering services to users over the network, for example, a voice conferencing server application, a social media application, and other applications as known.

In one or more embodiments, the programs 322 may provide platform that is web-based, or client-server based application via Web/API Unit 332 that provides for health research on a large population of subjects. The health platform may provide farmers the ability to conduct large N surveillance studies to map the incidence and prevalence of diseases within their herd of animals (i.e. subjects). The health platform may provide access for queries and data analysis of the farm database 328, the clinical database 330, and the subject database 334. The platform may allow for health analysis on different groups, including based on the subject's geographic information on the farm, genetic information, herd information, etc.

The prediction unit 324 receives farm data from a user device over a network at Web/API Unit 332, and/or from a third party database (not shown) and/or may operate a method to generate a prediction for the subject. The server may respond with the prediction to the user device, for example via a message from the Web/API Unit 332. The data may be stored in the farm database 328 along with the prediction data. Prediction unit 324 may determine predictive messages based on the model and the farm data.

The model generation unit 326 receives farm data from farm database 328, clinical data from clinical database 330, and subject information from subject database 334. The model generation unit 326 may generate a prediction model.

The farm database 328 may be a database for storing farm data received from the one or more user devices via Web/API Unit 332 and/or from third party databases. The farm database 328 may include livestock data.

Livestock data may include data associated with a livestock animal. For example, the livestock animal may be a dairy animal (i.e. an animal that is capable of production of milk), such as but not limited to a cow, a buffalo, a goat, or any other dairy animal that yields milk consumable by humans.

The clinical database 330 may be a database for storing medical record data, or other clinical data received from the one or more user devices via Web/API Unit 332. The clinical database 330 may include milk testing data for protein, fat, and somatic cell count in the milk of a subject of the herd. Measurements from a broad training population of subjects may be used to train the models generated by model generation unit 326.

The Web/API Unit 332 may be a web based application or Application Programming Interface (API) such as a REST (REpresentational State Transfer) API. The API may communicate in a format such as XML, JSON, or other interchange format.

The subject database 334 may be a database for storing subject information, including demographic information about each subject. Further, the subject database 334 may include the subject's nutritional information, genetic information, etc.

FIG. 3B shows a diagram of exemplary data sources 401a, 401b, 401c, 401d, 401e and 401f that can integrate with the systems and devices described herein. For example, as shown therein, source data from the third party data sources 401a, 401b, 401c, 401d, 401e and 401f may be collected to a centralized system 402, also referred to as a herd management software system, where the data is normalized, cleansed and then input to the server device(s) 300 described herein. There also may need to be validation between the different data sources 401a, 401b, 401c, 401d, 401e and 401f in order to create a unified data model, for example, if an animal is marked sold in one model but not in the other, or if there is disagreement in cattle identifiers.

The third party data sources may include, but not be limited to, feed sources 401a, the animal health related product sources 401b, milk component and milk quality measurement products 401c, milk depot products 401d, livestock sensor products 401e, and/or other data sources 401f.

For example, feed sources 401a may include feed management software products, such as but not limited to Feedwatch®, TMR Tracker and EZFeed that, for example, provide data regarding feeders to see how accurately rations are made and how accurately cows are fed.

For example, the animal health related product sources 401b may include animal health software products such as but not limited to Dairy Comp®, Bovisync™, PCDart™, MLK Group™, Delpro Horizon™ and GEA Dairy Net™ that, for example, provide data regarding the health of the livestock (e.g., dairy cows).

For example, milk component and milk quality measurement products 401c may include milk component and milk quality measurement software products such as but not limited to Afimilk™, Delpro Horizon™, GEA Dairy Net™ and DHI™ that provide data regarding components of milk and milk quality (e.g., fat content, protein content, volume, etc.).

For example, milk depot products 401d may include milk sales software products such as but not limited to Milk Board™, DFC™, Lala™, Land of Lakes™, and others that provide data regarding milk sales information and quantity.

For example, livestock sensor products 401e may include activity sensor software products such as but not limited to Heattime™, Allflex Livestock™, Intel™ and others that provide data regarding rumination, activity monitoring, in-line flow meters, livestock heat frequency and other breeding data.

For example, other data sources 401f may include environmental sensors that provide, for example, weather data, interior environment data (e.g., indoor temperature, humidity, etc.) or others.

Examples of each of these products and some of the different types of source data that they can provide to the centralized data server are shown in FIG. 3B. Data sources may provide data automatically (e.g., sensors that are present on the livestock and/or on the farm (e.g., temperature sensors, etc.). Data sources may also provide data manually (e.g., upon an input by a user, such as but not limited to milk production data).

FIG. 3B also shows that the systems described herein receive livestock data 403 from the centralized data server 402, also referred to herein as a herd management software system. The herd management system 402 is configured to receive the source data from each data source of a plurality of data sources 401a, 401b, 401c, 401d, 401e and 401f. The data sources 401a, 401b, 401c, 401d, 401e and 401f may be any of the data sources noted above, or any similar data source. The source data may include, among other types of data, health data, production data, growth data and genomic data for a herd of livestock (e.g., dairy cattle). Health data, production data, growth data and genomic data may be received from any one or more of the data sources 401a, 401b, 401c, 401d, 401e and 401f.

For example, the source data may include data collected from an ear tag attached to the livestock and/or a collar being worn by the livestock and/or bolus sensor. In this case, each of these devices may be configured to store any or all of health data, production data, growth data and genomic data and provide the health data, production data, growth data and genomic data to the herd management software system 402. Those skilled in the art will understand that ear tags worn by livestock and collars worn by livestock, and the data measured therewith and/or stored thereon is well understood in the art. For example, some ear tags can provide geographical location data, including but not limited to GPS data.

The source data received by the herd management system 402 is normalized to link the source data from each of the data sources 401a, 401b, 401c, 401d, 401e and 401f for a specified livestock of the herd and generate the livestock data 403. The livestock data 403 can be described as the source data from each of the sources 401a, 401b, 401c, 401d, 401e and 401f that has been organized in a database. For example, the source data from each of the data sources may be organized by creating tables and establishing relationships between those tables according to rules designed both to protect the data and to make the database more flexible by eliminating redundancies and inconsistent dependencies.

Once the livestock data has been received by the system and devices described herein, a data model based on the livestock data 403 may be generated or the consolidated data pool may be used by machine-learning algorithms to generate data models on demand. Generating the data model may include, but is not limited to including, creating a visual representation of either a whole information system or parts of it to communicate connections between data points and structures.

For example, as is described in greater detail below, the data models that may be generated herein include, but are not limited to, lactation curves, feed conversion, growth rates, impact of weather on animal health and/or milk quality, and somatic cell count levels.

The data models generated by the systems described herein identify a trend in the health data of the herd of livestock. For example, in one embodiment, the data models may identify that a disease is passing though the livestock. They may also relate to production data (i.e., milk output), milk components (i.e., level of fat, proteins, etc. in the milk produced by the livestock), somatic cell count or other milk quality data, growth rates, and body condition scoring and lameness information, for example.

Based on the data model and the identified trend, the system and devices described herein can then generate a task for managing the herd of livestock. For example, returning to the example of the data model identifying that a disease is passing though the livestock, the systems and methods described herein may generate a treatment regime for the livestock. Tasks may also include vaccine administration, for example.

The tasks generated can be implemented by one or more protocols that may also be generated by the systems and devices described herein and provided to users, by way of, for example, the user interface of one or more of the mobile devices and/or computing devices and/or display devices described herein. The protocols may be provided on the server by a provider of the systems (i.e., pre-populated) and/or customized by a user of the system (i.e., populated by the user). For example, the tasks generated may relate to milking a livestock (e.g., cow) and the protocols may provide instructions on how to milk a cow. The instructions may include, but should not be limited to including, video instructions (e.g., a link to a video hosted by a third party or a direct video can be provided), audio instructions (e.g., a voice message that can be associated with the video or unassociated with the video can be provided, again, either as a direct audio message or a link to an audio recording hosted by a third party), written instructions describing how to complete the task and/or pictures showing how to complete the task. The instructions may relate and/or provide standard operating procedures (SOPs). If the protocols are populated by the user, the user may upload the video instructions, audio instructions, written instructions and/or pictures to the devices described herein for them to be provided to other users, for example in response to the task noted in the protocol being generated. These tasks and protocols are available for use in other areas of the application as well.

In one embodiment, a data source may provide health data or the livestock. Herein, the term health data refers to health conditions, reproductive outcomes, causes of death, and quality of life of a livestock. For example, health data may include cow calendar information, such as but not limited to a date that the livestock had a calf and/or a date that the livestock was bred. Health data may also include an age of the livestock (e.g., their birth date), how many pregnancies the livestock has had, t

Herein, the term production data refers to information pertaining to a substance produced by the livestock, such as but not limited to milk produced by dairy cattle. Production data may include volume, quality measures or other information.

Herein, the term growth data refers to information about the age and/or size of the livestock, such as but not limited to weight and increase in weight over time.

Herein, the term genomic data refers to information regarding the DNA of the livestock, including but not limited to lineage data.

Referring now to FIG. 4A, shown therein is one example of a user interface 400 of the platform as shown on one of the computer devices 102.

The systems and methods described herein offer several features that provide for events to be stored, associated and triggered based on an image that will provide context to those events. Similar to a dental record showing a pictorial representation of teeth in a mouth, the systems, devices and methods described herein include several features for using a pictorial representation, such as an image depicting a geography and building layout of a farm, an image of a cow hoof or an udder, or another portion of a body of an animal. For example, FIG. 4B shows parts of an animal and FIG. 4C shows a cow, each having different regions of the object in the image partitioned into sections for identification purposes.

The systems, devices and methods described herein provide for data inputting that is carried out in a way that the data can be associated with a selected area of the image. The systems, devices and methods described herein we can also create visually apparent triggers when a threshold value of the data associated with a specific area of the image is met or exceeded, or when the interaction of multiple areas that are related hit a threshold.

For example, FIG. 4A shows various farm data collected from subjects in identified pens of a farm. Each pen on the farm is identified (e.g. by a reference number). Data associated with each pen is presented within a user-defined boundary (e.g., box or drawn polygon) overlaying an aerial photograph of the farm showing a location of each of the pens where animals are housed.

For example, using the systems, devices and methods described herein, a user can collect an aerial photograph, a two-dimensional, three-dimensional or four-dimensional, artificial reality, building plan, AutoCAD output or drawing, or the like, of their farm and upload the photograph, or other image, to a server hosting the platform described herein. The photograph or other image will preferably include enough resolution—or a drawing that would show the barn layout—for the user to indicate a location of each of the pens of the farm on the photograph. A user then uses the platform to draw a box on the approximate geographic location of each pen on the photograph. It should be noted that the user-defined box can be of any shape, including but not limited to square shapes and non-square shapes. The user can then use the system to assign herd data, either collected using the systems described herein or migrated into the systems described herein from another software platform (e.g. Dairy Comp), to one of the selected geographic areas on the photograph. For example, specified livestock data (i.e., data that is specific to one of the livestock) can be assigned to the selected geographic area, the specified livestock data including location data providing a geographical location of the livestock.

The systems, devices and methods described herein can also support integration of third-party mapping services and frameworks, allowing users to utilize existing aerial maps without needing to capture their own photographs.

For example, a user can draw a boundary on the photograph using the systems described herein and associate the boundary to a pen of cattle, or another animal, on the farm. The systems described herein then populate various data structures to be presented on the photograph based on the data of the cattle in that pen.

For example, data structures may present the numbers of cows in the pen, the number of cows that are pregnant and in each pen, the number of cows that are milking (i.e., fresh) and number of cows that are open (i.e., non-pregnant), number of cows with a task or alert. Images may also be used to indicate trends and warnings.

Each cow in each pen may be identified based on subject data, such as but not limited to subject data stored on an ear tag of the cow or another identifier. Subject data associated with each cow tag may include the name of the cow, the age of the cow, data associate with the mother of the cow, data associated with the father of the cow, etc.

The systems and methods described herein may be configured to associate health data of each cow with its subject data. For example, health data may include somatic cell count, protein and/or fat present after measuring samples of milk taken from the associated cow.

Health data, production data, growth data and genomic data as noted above may be used as an indicator for one or more issues such as health problems in dairy cattle and/or degradation in milk quality/quantity from a cow. For example, high somatic cell counts may indicate that poorer milk quality/quantity. The aforementioned features may be used to compare health data of cows in a same pen and/or in different pens.

In another embodiment, locations on the map or image may also indicate tasks that need to be done in that designated area/workstation and could (for example, in real-time) show real-time locations of team members, animals, hardware and equipment and other needed resources. In one embodiment, the locations of the real-time locations of team members (i.e., users of the software), animals, hardware and equipment and other needed resources may be provided by the GPS co-ordinates of a mobile device, such as but not limited to a mobile phone, or by a tracking device that uses wireless technology to report its location relative to other devices, such as but not limited to an Apple AirTag®.

In another embodiment, tasks may be assigned to team members based on their location. For example, tasks may be assigned to team members based on their location in combination with qualities team members and/or capabilities or skill set of the equipment that they are near. For example, a task may be assigned to a team member if the team member is proximate to a piece of equipment that there are qualified to operate.

In at least one embodiment, multiple images and/or drawings uploaded to the system drawings can be stitched together (i.e., a three-dimensional overview of the farm can have a two-dimensional AutoCAD® image overlaid over a building.

In at least one embodiment, the aforementioned boundary boxes (or polygons) drawn over images can be assigned groupings. For example, the boundary boxes may be assigned to calf hutches, buildings, feed bunks, workstations, etc. In at least one embodiment, the user (e.g., by the user device) could be provided with data or actionable insights for types of groups. For example, a number of calves in hutches could be presented.

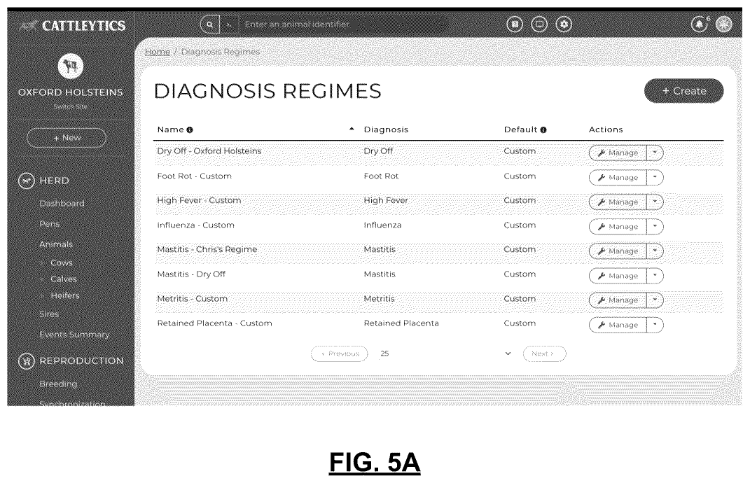

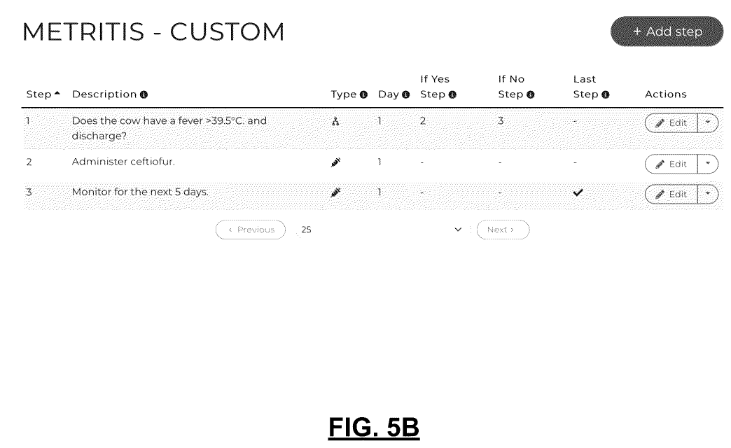

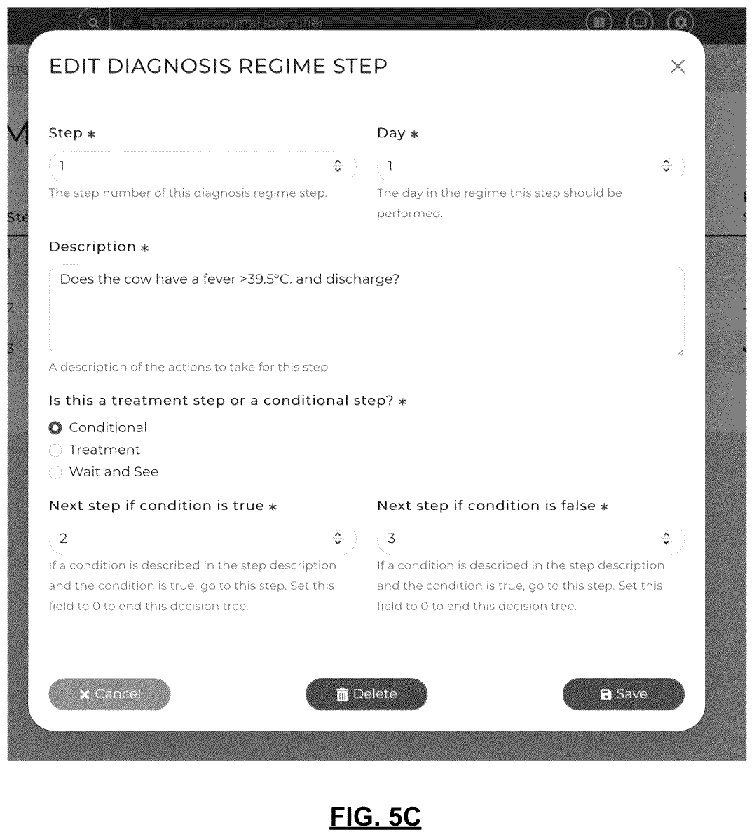

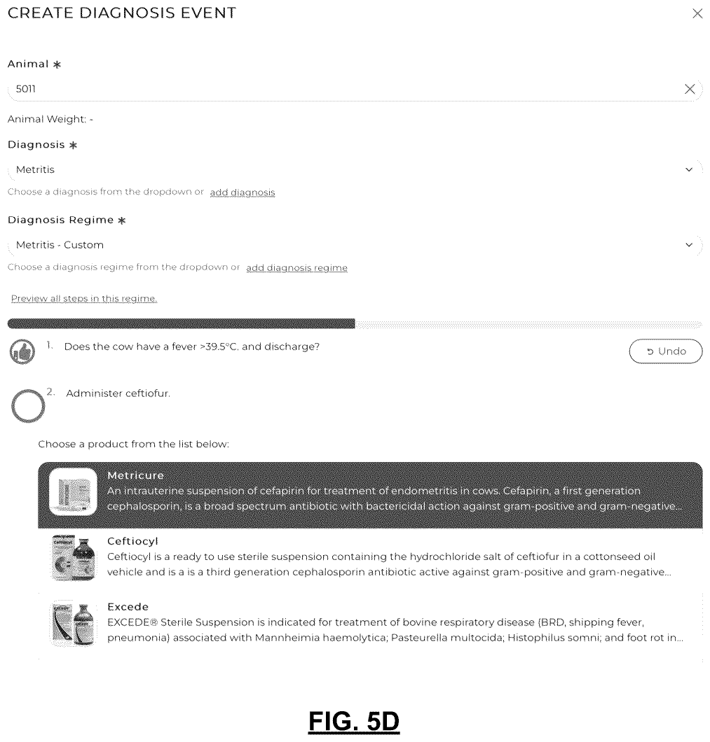

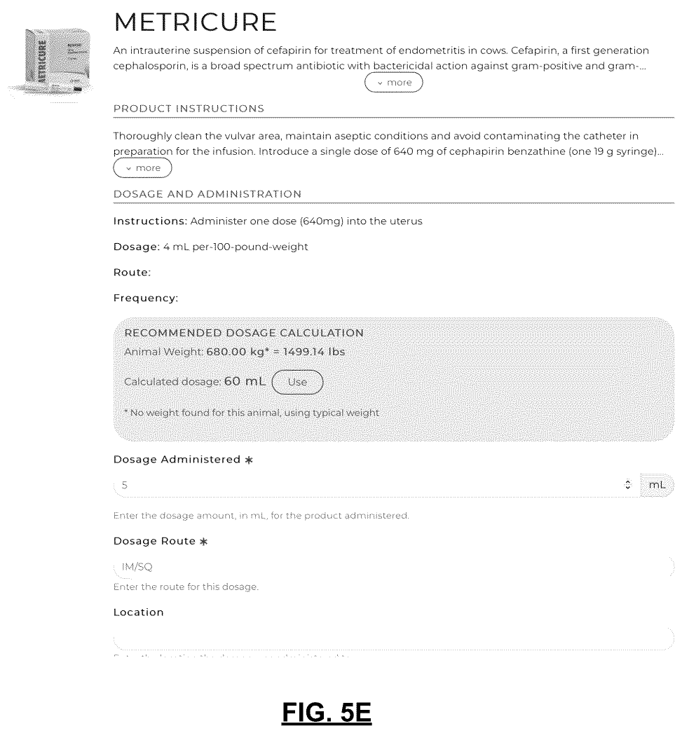

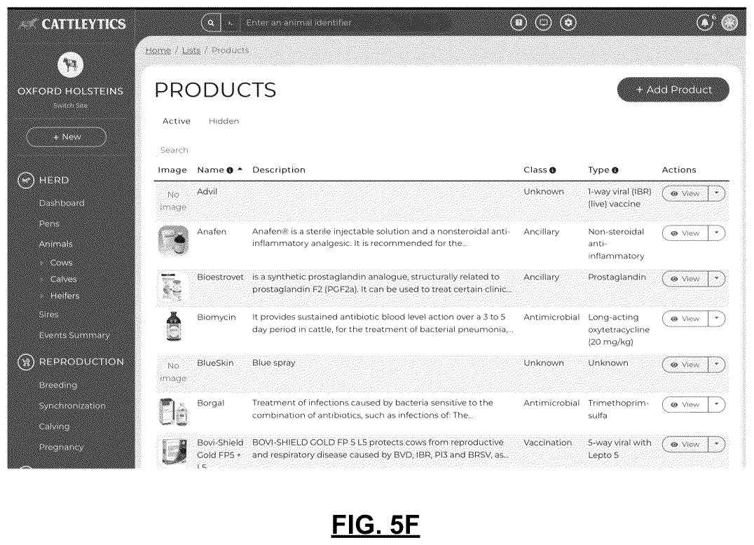

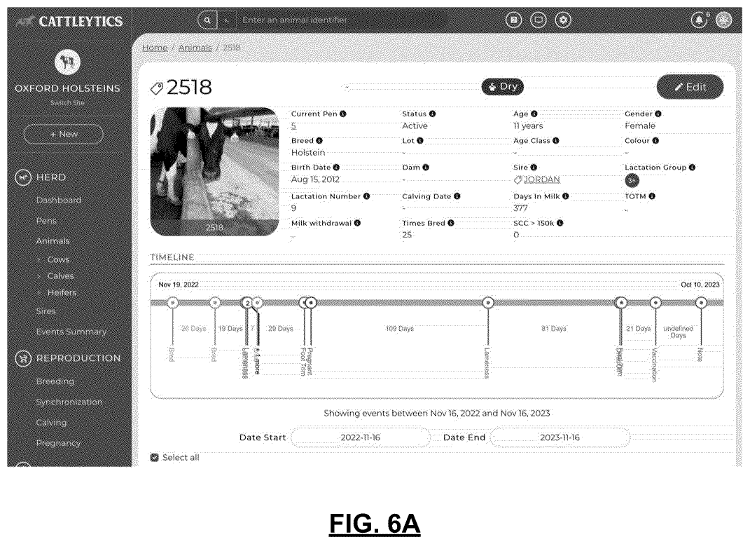

Turning now to FIGS. 5A-5F, shown therein are example user interfaces of a treatment decision tree feature of the platforms described herein. The systems and methods described herein are configured to present a platform where pre-set and customizable diagnosis regimes, for example for dairy cattle diseases, can be stored and/or generated automatically. Users can create and/or customize treatment decision trees, including timing of events (e.g. when medication is given) and dosages to be administered.

In at least one embodiment, the systems and methods described herein are configured to develop customize treatment decision trees by, for example, analyzing data stored in the systems and identifying trends. The systems and methods are then configured to offer treatment recommendations based on collected information and/or farm data, such as but not limited to subject data.

In at least one embodiment, users can also create a treatment decision-tree regime that would use yes or no questions, or request values to be entered (e.g., temperature, number of previous treatments), and then conditional statements to guide a user through the decision-making process when treating an animal. These can also include peripheral devices gathering information (e.g., temperature, weight, or the like) and using those to generate treatment decisions. This may or may not implement machine learning in order to continue to modify the best decisions based on previous outcomes.

The decision tree may include questions, such as but not limited to yes/no questions, scoring questions, etc. Responses from a user flow into certain branches of the decision tree presented on the UI. Certain steps of the decision tree may rely on timing and dosages provided to individual livestock. Certain steps may also create a task for follow up, for example at a future point in time. For example, the task may be to monitor the livestock and not include an active step of treatment.

Other types of decision questions include, but are not limited to, ranges, multiple choice, scale, and external peripheral questions. For example, the questions may ask the user to report a weight range of the livestock and the task presented to the user, and/or follow-up questions, may vary based on the response of the user.

For example, as shown in FIG. 5A, dosage regimes can be created and stored for a variety of diseases, such as but not limited to pneumonia, mastitis, metritis, retained placenta, etc. They can also be used for preventative treatments and general cow health maintenance regimes such as hoof trimming and ketosis testing.

For example, the systems described herein may suggest drugs for treatment of the one or more diseases (or ailments) based on measured temperatures of the subject and other subject data, such as but not limited to weight of the subject, gender of the subject, age of the subject, or the like. The system may use machine learning in order to make the best assessment according to the history of similar subjects and their responsiveness to a given treatment or prevention strategy.

A user may input a drug type, a quantity, and a time, for example, into the platform and track administration of the drug over time. In at least some embodiments, the models generated and stored in the systems described herein may be used to compare treatment efficacy of different drugs, or lack of treatment, for example, or other factors in the decision trees, across one or more farms.

FIG. 5B shows an exemplary regime for the diagnosis of metritis. Here, a treatment decision-tree regime including at least three yes or no questions is shown for a user to consider when they suspect that an animal is suffering from metritis. Conditional statements guide user through the decision-making process when treating an animal. There can be several different protocol statement types such as “treatment”, “wait and see”, and a specialized final step.

Diagnosis regimes can be created in different ways, such as by a third party, such as a veterinary practice, be pre-populated into the system as general best practices, or created by the farmer themselves. They can also be set up as templates to allow users to customize the regimes. As shown in FIG. 5C, each diagnosis regime is editable by the user. For example, the user may create a diagnosis event (see FIG. 5D) including, for example, the administration of a drug product. The system may be configured to present the user with a list of drug products that are commonly used in the treatment of diseases of the animal or those that are on-label for the animal in a particular geography. The dosage of one or more drugs can be recommended by the system based, for example, on subject data of the animal (see FIG. 5E) or using on-label pharmaceutical dosages for a given country. As such, these regimes can be set up to be geographically sensitive. Other information regarding the administration of the drug can also be presented to the user, such as but not limited to the dosage route of the drug and the physical location that the drug is stored on the farm. In at least one embodiment, system may link the various treatment plans to a drug inventory of the farm. For example, the user may manually enter a quantity of each of various drugs to be stored in the systems described herein. Then, as a user administers a quantity of a drug to an animal, a quantity of the drug stored in the inventory can be automatically reduced. The user can also scan a drug to connect it to the drug inventory in order to also make use of information such as expiry dates.

FIG. 5F shows an example of a drug inventory of a farm including a list of available drugs to be administered to animals.

In at least one embodiment, the systems described herein can may integrate treatment plans for one or more animals into a calendar management tool, for example setting tasks for the user to perform over a period of time including, for example, storing tasks including dosages of drugs to be administered to specific animals on specific days to implement the treatment plans described herein.

In one embodiment, models that are generated by the systems and devices described herein can be combined. Combining the models may help users, such as but not limited to cattle farmers, with resource management. For example, indicating the amount of animals freshening, eligible for breeding, or benefiting from preventive actions during their life, etc. can be tied into generated or recurring tasks for workers on the farm but can also be generated for consultants and industry partners. For example, models indicating lower milk outputs could generate a task for a local feed company to take a bunk sample for testing to ensure quality and dry matter values are as expected or if there is a need for a modification in their rations. Combined models can be used to predict and/or assist with having resources available on the farm. For example, resources like labor, supplies, feed, sensors, animal slots in designated areas can be accurately tracked and predicted using the systems and methods described herein. Predicting the availability of farm resources such as those noted above ahead of time can help a farm anticipate early on reducing cost and increasing efficiency by having all resources available when needed.

In at least one embodiment, the models described herein may learn from previous years data and may use time series forecasting or similar machine learning models. For example, when weather gets more humid, more calf workers may be scheduled to follow up on health challenges since previous years learned incidence of health related issues would increase by X %.

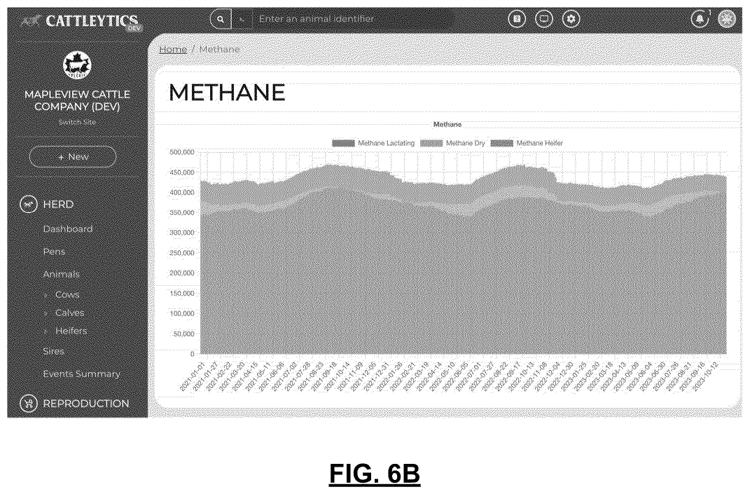

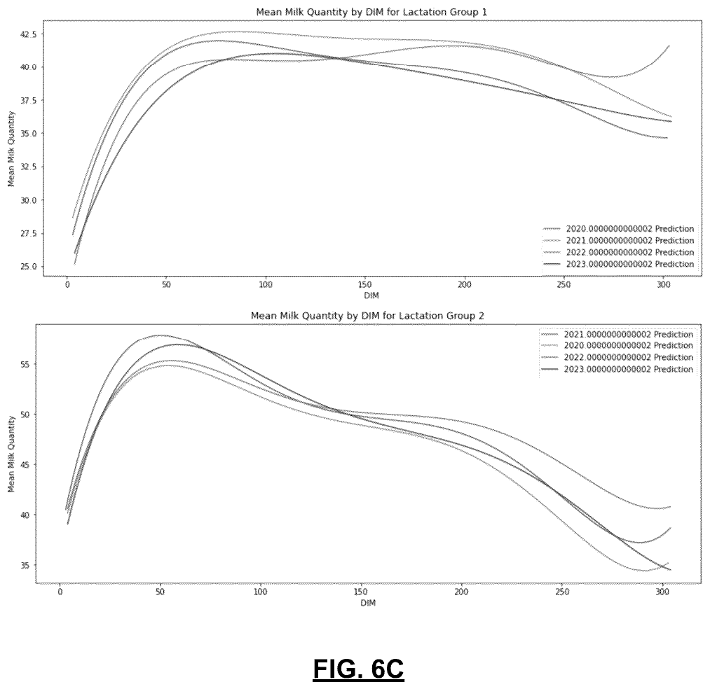

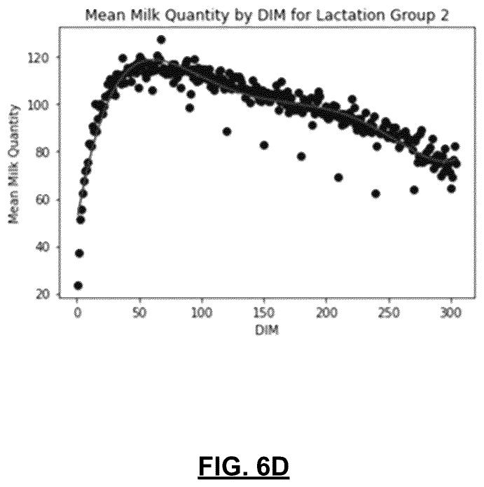

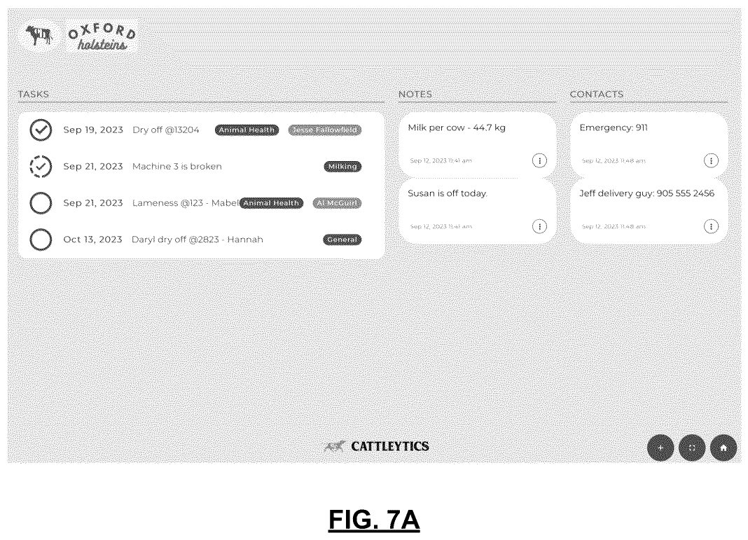

Turning now to FIGS. 6A-6D, the systems described herein may be configured to generate personalized timelines for each animal in a herd. The personal timelines may include past events in the animals life (including but not limited to birth date, date bred, date of lameness, date of foot trim, date of pregnancy, date of vaccination, etc.). An example of a timeline is shown in FIG. 6A.

The personal timelines may also include dates of predicted events, such as but not limited to a date of giving birth and/or lactation period start and end dates.

As dairy animals lactate, monthly milk testing or on-farm systems that can gather daily data for protein, fat, and somatic cell count, for example, is standard. Dashboards of the systems described herein visualize trends, for example trends of protein, fat, and somatic cell count in a herd of dairy cattle, and establish baselines, helping users make informed decisions based on historical data. The animal timelines may also combine visualized data from data sources like these as well as other sources, such as milk quantity, somatic cell count, growth rates, rumination and activity rates so that these values can be correlated with life events.

In at least one embodiment described herein, the systems and methods described herein store models that can be compared to subject data and predict future trends and behaviors, such as milk volume and herd size, based on subject data such as milk quality, protein, fat, and somatic cell count as well as other subject data, such as historic data (e.g. number of pregnancies, genetic information, etc.). These insights may be used to inform practical decisions, for example choosing breeding strategies or using embryo transfer for faster genetic changes. The models can also use multi-farm data to predict milk quantity, including milk components like fat, protein and total milk solids, in order to make supply predictions for the group of farms.

For example, the models use historical data and can set up a baseline for a specific animal, a group of animals, or a herd on a farm, for example, to both make it apparent when any specified animal, group of animals, or herd is departing from their modelled baseline, and how their predicted model baseline would impact future outputs.

For example, the models can collect historical milk quality test data from each animal in a herd and create a polynomial equation for milk quantity specific to that herd. This could be completed, for example, for each lactation of the herd (typically lasting about 305 days). Individual cow data can be compared to the linear regression model to assess milk quality.

Subject data collected about the individual, environment or situation can be collected from many different sources so that models of the systems described herein can be used to suggest future behaviour. These data points may include but are not limited to cow data (e.g., age, weight, genetics, feed type, etc.) and non-cow data factors (e.g. local weather data to plot temperature or humidity by month to somatic cell counts). Data models described herein are customized based on the subject data for each farm. Large aggregate data sets can also be used in conjunction with these to create a base data model that can be used to add confidence and trending for smaller farm datasets.

The system may also use machine learning to look at multiple factors at once and make predictions with respect to milk volume produced, herd size, etc.

Information predicted, for example regarding future milk volume based on predicted herd size, can be valuable in jurisdictions (such as Canada) where each farm has a quota of butterfat that it may produce. If a herd produces too much butterfat, and secondarily milk, the farm may be charged for the surplus. This can also be used for aggregated farm predictions so that national and local level milk supply values can be predicted and better modified to match demand.

The above-noted models and suggestions based on the models can then be tied to implementations for the farmer on the farm. For example, if the models indicate that the farm will not need additional milk producing cows in the future (e.g. because its predicted volume of milk produced will meet or exceed its quota), the user can choose to alter its breeding strategy. For example, the user may choose to breed beef cows (with beef semen). In another example, if predicted milk volumes are less than the farm's quota, the systems described herein may suggest to the farmer to breed its animals with sexed semen to ensure that any calves that are born are female (if more milk is going to be necessary). Further, the models may indicate that the user should use an embryo transfer to more quickly change the genetics of a specific animal, for example. These decisions can be made based on the predictions and models described above.

Referring to FIG. 6B, shown therein is a methane emissions graph created using a methodology described herein. The methane emissions of a herd is shown over time. It is generally understood that boosting milk-production efficiency can reduce cow methane-emission intensity. Using genomic testing, and combining estimated data for those animals sired by low-methane bulls, this additional information can be used to better predict the methane output of these animals.

The systems and methods described herein may include at least the following steps:
1. Collect available data from the farm tied to production, such as but not limited to the volume of milk, fat and protein produced by each of the cows on the farm.
2. This collected data, can be used to create one or more custom equations to represent a biological system, environmental system or various systems on the farm. These systems models can be stratified by the parameters that have the biggest impact on them, to create customized models to best represent specific items such as animals, equipment or environmental situations (e.g. for different lactation numbers, production outputs look very different).
3. Using those equations to smooth information gaps, such as gaps in milk production data when the farm only collects data monthly for most animals on the farm (difficult to create a lactation curve).
4. Parameterizing or stratifying those equations to fit individual animals.
5. Using new estimated data points based on the equations to generate models such as fat and protein corrected milk production estimates and, for example, tying the milk production data to equations and concepts such as but not limited to methane gas emissions.

FIG. 6C shows exemplary modelling output for milk production based on the examples described herein.

FIG. 6D shows exemplary mean milk production quantity by days in milk (DIM) calculated using the exemplary polynomial equation shown therein.

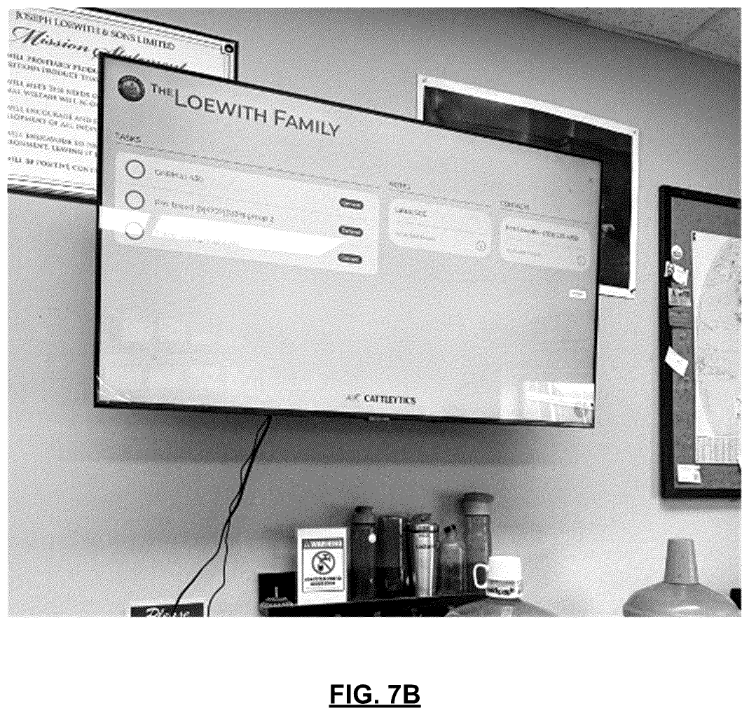

Turning now to FIGS. 7A and 7B, task management of the above-noted systems can be presented in a centralized manner on a display device or kiosk, also referred to herein as a digital whiteboard. An example of a task management UI of the display device is shown in FIG. 7A. The UI of FIG. 7A may be presented, for example, on a display device that is centrally located on the farm, such as but not limited to in an office of the farm, for example as shown in FIG. 7B.

The UI of the digital whiteboard can present tasks to be completed, for example on one or more days, as well as notes for the users of the system and contact information of third parties that may be interacting with users of the system. It may also display weather data, staff scheduling data and items such as active treatments. The UI of the digital whiteboard may be centralized such that users with mobile devices can add tasks using their mobile devices that are then presented centrally on a central display device (such as is shown in FIG. 7B). As tasks are completed, users can either use a kiosk physically connected to the central display device or can use a mobile device to indicate tasks have been completed. Tasks may also be visualized in such a way that items in the queue to be completed, items that are in progress, items that require review, and items that are completed can be displayed.

Information to be displayed on the display device can be selected by users and can include tasks, contact information, notes, etc. such as but not limited to the task(s) generated by the systems described herein. Tasks can also be identified as being started by users and their status can be centrally updated on the whiteboard. Other key information for the dairy, including current ongoing treatments, withdrawal dates and contact information can be presented on the digital whiteboard. There is also the ability to toggle to a visual representation of the farm to allow for farm-level image-based data modelling to be shown. Other information that may be presented on the UI of the display device may include user input from a user device (e.g., in the form of notes), weather information, a calendar and/or scheduling information, current staffing (e.g., other user information). In at least one embodiment, users of the system have a same or similar user interface presented on their user devices as are presented on the digital whiteboard.

In another embodiment, the tasks that are presented on the display device may be single, (i.e., one-off) tasks or can be recurring tasks that are performed in a farm environment.

In another embodiment, data from external sources can be displayed on the display device, such as but not limited to weather data, camera views, and/or data from other devices/sensors on farm.

In another embodiment, data that is presented on the display device can be customized, for example based on the geographical location of the display device and/or based on the capabilities and/or skill sets of the users in proximity of the display device, the time of day, etc. For example, the UI of the display device may present a Notes section that may present tasks that are specific to users that are logged into the software on user devices that are proximate to the digital display. based, or time of the day/scheduled. This device can also use facial recognition to validate the user and show information specific to that user.

In another embodiment, the systems, devices and methods described herein may include one or more emergency alerts. In one embodiment, the emergency alert may be triggered based on a location of one or more of the users and/or livestock. For example, an emergency alert may be generated based on a location of a livestock relative to a user-defined boundary (e.g., if a livestock leaves a pen). Other alerts can also be programmed and triggered, for example if a water system detects freezing or excessive use. Alert(s) may also be triggered on user devices based on their role on the farm, skill set, location, etc. These alerts may also come in the form of notes requiring an acknowledgement of receipt to allow management to ascertain that all workers have viewed important alert information.

The systems and methods described herein provide for farmers, such as but not limited to dairy farmers, to forecast and change the future of their dairies. By inputting their local goals and constraints they can visualize their future outcomes based on their current trajectory, craft a new goal-based trajectory, and develop data-driven integrated processes to reach those goals. The systems and methods described herein address complex factors impacting future milk output, like the impact of current pregnancies, and the challenges of accessing feed cost data in an automated way. The systems and methods described herein may be used to model dairy economics in a manner that is not currently available.

The systems and methods described herein include a comprehensive cloud-based dairy management application, that provide for farmers to be empowered to make data-driven decisions by having complex data distilled from multiple manual and automated data inputs synthesized in an intuitive, customizable and actionable data and visualization.

The systems and methods described herein introduces a dynamic dashboard for economic modelling designed for dairy farms. It also introduces triggerable actions so that when a condition is reached, a course of action is suggested. An example of this would be milk dropping below a percentage threshold, and investigations into specific animals, or a ration change may be suggested. This could also be animals to be dried off based on a combination of milk production and days to calving. These suggestions may also be optimized by integrating multiple system datapoints into a machine learning algorithm. It also suggests action. based on external data such as market information or available genomics, so that if there is an opportunity available, the user can be made aware of this. An example of this would be a low commodity price for a part of a ration that would result in more profitable milk production if it was used as an input. This could also include a new drug that could minimize treatment cost and/or improve efficacy.

In at least one embodiment, the systems and methods described herein offer a bilingual (English and French) user interface and may include other languages and globalization options.

In at least one embodiment, the systems and methods described herein offer side-by-side comparison of different treatment regimes, or a treatment versus no treatment comparison.

In at least one embodiment, the systems and methods described herein offer comprehensive records to support the Canadian Quality Milk initiative or comparable initiatives in other countries.

In at least one embodiment, the systems and methods described herein offer measurement and reporting on the impact of utilizing sustainable practices and resulting improvements in the farm's or farm group's carbon footprint. This data can also be integrated with systems such as the open-source Holos carbon footprint system in order to calculate an overall farm carbon-footprint.

In at least one embodiment, the systems and methods described herein offer integration with co-developed data pipelines, pulling in multiple dairy data sources.

In at least one embodiment, the systems and methods described herein offer hassle-free interoperability with third party tools and software.

In at least one embodiment, the systems and methods described herein offer farmers the ability to look ahead, with planning tools that can be coupled with expert opinion such as their veterinarian, economist or nutritionist to design and monitor SOPs and protocols, as well as the opportunity to reflect, monitor adherence and see how successful a practice has been, making dairy farms more competitive in an increasingly data-driven agricultural world. This aggregated data can also be used in a similar but grouped manner for government, veterinarians and veterinary groups, nutritionists and feed groups, and other similar industry and governmental organizations.

In at least one embodiment, the systems and methods described herein may provide improvements in profitability, sustainability, animal health, and animal welfare.

The present invention has been described here by way of example only. Various modification and variations may be made to these exemplary embodiments without departing from the spirit and scope of the invention, which is limited only by the appended claims.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

What is claimed is:

1. A system for managing a herd of livestock, the system comprising:
a memory;
a processor in communication with the memory, the processor configured to:
receive livestock data from a herd management software system, the herd management system configured to:
receive source data from each data source of a plurality of data sources, the source data including at least one of health data, production data, growth data and genomic data for the herd of livestock; and
normalize the source data received from each data source of the plurality of data sources to link the source data from each of the data sources for a specified livestock of the herd and generate the livestock data;
create a data model based on the livestock data, the data model identifying a trend in the health data of the herd of livestock;
generate, based on the data model and the identified trend, a task for managing the herd of livestock; and
output the task.

2. The system of claim 1, wherein the health data includes cow calendar data for each livestock of the herd.

3. The system of claim 1, wherein the generated task is a treatment plan for one or more specific livestock of the herd or a vaccine administration timeline.

4. The system of claim 1, wherein the data model includes a personalized timeline for each livestock in the herd, the personalized timeline including past events during the life of the livestock including at least one of: birth date, date bred, date of lameness, date of foot trim, date of pregnancy, and date of vaccination.

5. The system of claim 4, wherein the personalized timeline includes one or more dates of predicted events, the predicted events including one or more of a date of giving birth and/or a lactation period start date and/or a lactation period end date, the predicted events being determined based on the health data.

6. The system of claim 1, wherein the livestock data includes production data and the data model includes a model of milk production on the farm.

7. The system of claim 1, wherein the processor is further configured to, after generating the task for managing the herd of livestock, generating a protocol for completing the task.

8. The system of claim 7, wherein the protocol includes video instructions, audio instructions and/or written instructions for completing the task.

9. The system of claim 7, wherein the protocol is pre-populated by an operator of the processor.

10. The system of claim 7, wherein the protocol is at least partially provided by the user by uploading at least one of the video instructions, the audio instructions and/or the written instructions to the processor before the processor generates the task.

11. The system of claim 1, wherein the processor is further configured to:
receive image data depicting at least a portion of a farm;
receive a user input indicating a boundary of one or more selected geographical areas of the farm;
receive a selection of one or more livestock of the herd, the one or more livestock having specified livestock data associated therewith stored on the memory; and
assign the one or more livestock to the selected geographical area of the farm.

12. The system of claim 1, wherein the specified livestock data includes location data providing a geographical location of the livestock.

13. The system of claim 11, wherein the image data is one of an aerial image, an AutoCAD drawing and mapping data received from a third party data source.

14. The system of claim 11, wherein the processor is further configured to display the specified livestock data on a user interface depicting the boundary on the image data, the specified livestock data overlaying the image data within the boundary.

15. The system of claim 1, wherein the task includes a treatment regime for an illness of the herd, the illness being identified based on the identified trend.

16. The system of claim 1, wherein the treatment regime includes a suggestion of a drug to administer to the livestock to treat the illness, the suggestion based on the health data, the health data including at least one of: a temperature of the livestock, a weight of the livestock, a gender of the livestock and an age of the livestock.

17. The system of claim 11, wherein the treatment regime includes a dosage route of the drug and/or a physical location of the drug on the farm.

18. The system of claim 12, wherein the treatment regime includes an inventory of the drug on the farm.

19. The system of claim 1 further comprising a display device centrally located on the farm, the display device presenting the task to be performed to manage the herd of livestock.

20. A computer implemented method of managing a herd of livestock, the method comprising:
receiving, at a processor, livestock data from a herd management software system, the herd management system configured to:
receive source data from each data source of a plurality of data sources, the source data including health data for the herd of livestock; and
normalize the source data received from each data source of the plurality of data sources to link the source data from each of the data sources for a specified livestock of the herd and generate the livestock data;
creating, by the processor, a data model based on the livestock data, the data model identifying a trend in the health data of the herd of livestock;
generating, by the processor, based on the data model and the identified trend, a task for managing the herd of livestock; and
outputting the task.
